(19) United States
(12) Patent Application Publication
(10) Pub. No.: US 2003/0059943 A1
(43) Pub. Date: Mar. 27, 2003

(54) Inhibition of expression of a target gene

(76) Inventors: Ulrich Certa, Allschwil (CH); Kenneth Lundstrom, Oberwil (CH)

Correspondence Address: HOFFMANN-LA ROCHE INC., PATENT LAW DEPARTMENT, 340 KINGSLAND STREET, NUTLEY, NJ, 07110

(21) Appl. No.: 09/994,412

(22) Filed: Nov. 27, 2001

(30) Foreign Application Priority Data
Nov. 29, 2000 (EP) 00126113.0

(51) Int. Cl. A61K 48/00; C12N 15/86

(52) U.S. Cl. 435/456; 424/93.2

(57) ABSTRACT

The present invention relates to a process for inhibiting the expression of a target gene in cells or tissue by infecting said cells or tissue with viral particles consisting essentially of ss RNA which expresses a sense RNA strand, and viral particles consisting essentially of ss RNA which expresses an anti-sense RNA strand, wherein the sense and anti-sense RNA strands comprise homologous nucleotide sequences to a portion of the target gene.

### BACKGROUND OF THE INVENTION

The inhibition of gene expression impacts on therapeutic research.

Classical genetic techniques have been used to characterize mutations in organisms with reduced expression of selected genes. Such techniques require laborious screening programs and have been limited to organisms in which genetic manipulation has been already established.

These difficulties may be overcome by a method of using double stranded (ds) RNA interference to inhibit gene expression in mammalian cells. The technique is based on the delivery of ds RNAs into cells, where interference with specific messenger RNA (mRNA) molecules will occur to inhibit gene expression.

In the International Patent Application WO 99/32619, a method to inhibit specifically gene expression in an invertebrate model organism is described. This method is based on the use of ds RNAs and their introduction into a living cell to inhibit gene expression of a target gene in that cell. The ds RNAs are introduced into the cell, i.e. intracellularly, or extracellularly, i.e. within a body cavity.

In the international patent application WO 99/32619, the use of a viral construct packaged into a viral particle may be efficient for introduction of an expression construct into the cell and the transcription of RNA encoded by the expression construct.

Constructs with both sense and anti-sense sequences in the same viral vector did not successfully inhibit gene expression, most likely due to inefficient interaction with target mRNA. It was postulated that when the sense and the anti-sense RNAs are encoded by one construct, the RNA duplex formation occurs immediately and no interaction with mRNAs is possible.

More recently, a scientific publication (F. Wianny and M. Zernicka-Goetz, Specific interference with gene function by double stranded RNA in early mouse development, Nature Cell Biology, vol. 2, February 2000, pp. 70-75) described that synthetic ds RNAs were introduced into both mouse oocytes, and preimplantation of embryos was carried out by microinjection. The publication reported that specific inhibition of gene expression was achieved.

One major difficulty is, at present, the delivery of the ds RNA into cells efficiently. No genetic technique in this domain has been developed for direct introduction of ds RNAs into cells.

It would be useful to develop a technique to specifically inhibit the function of individual genes. The ability to inhibit a specific target gene without affecting other genes of the cell would be of great importance.

It would also be useful to prevent the progression of specific diseases, like cancers, infectious diseases or neurological disorders by inhibiting the function of specific genes, for example.

It would also be useful to be able to analyze the differences between normal and diseased tissue.

Clearly, introducing ds RNAs into cells biologically rather than mechanically would be beneficial. Such introduction would reduce manipulations and circumvent the generation of mechanical cell damage.

It would be of advantage for the study of cell proliferation, to analyze gene function or the functional alteration of gene expression. Certain genes may be required for cell or organism viability at only particular stages of the development.

The ability to inhibit a specific target gene at a specific time and at a defined location in tissue or organisms without introduction of permanent mutations into the target genome would be of substantial interest.

### SUMMARY OF THE INVENTION

The present invention provides a process to inhibit the expression of a target gene in cells or tissue comprising infection of the cells or tissue with (a) viral particles consisting essentially of single stranded ribonucleic acid (ss RNA) which expresses a sense RNA strand and (b) viral particles consisting essentially of single stranded ribonucleic acid (ss RNA) which expresses an anti-sense RNA strand, wherein the sense and anti-sense RNA strands comprise homologous nucleotide sequences to a portion of the target gene. The two complementary RNA strands interfere with the expression of a target gene in a cell.

### BRIEF DESCRIPTION OF THE DRAWINGS

FIG. 1: Inhibition of Aldolase A expression with block stocks.

Panel A: Schematic representation of the human aldolase A gene and probes used for expression analysis. A and B are fragments used to probe Northern blots (FIG. 2). V and G are primers pairs that amplify either RNA expressed by the virals stocks or else selectively chromosomal transcripts.

Panel B: Detection of either virally encoded aldolase mRNA with VA/VB (first two pictures form top) or endogenously encoded mRNA using (GA/GB lower panels). Co are unifected control cells, s is infected with the sense strand virus and as with the anti-sense virus and ds represents a 1:1 mixture of both virus stocks (block stock).

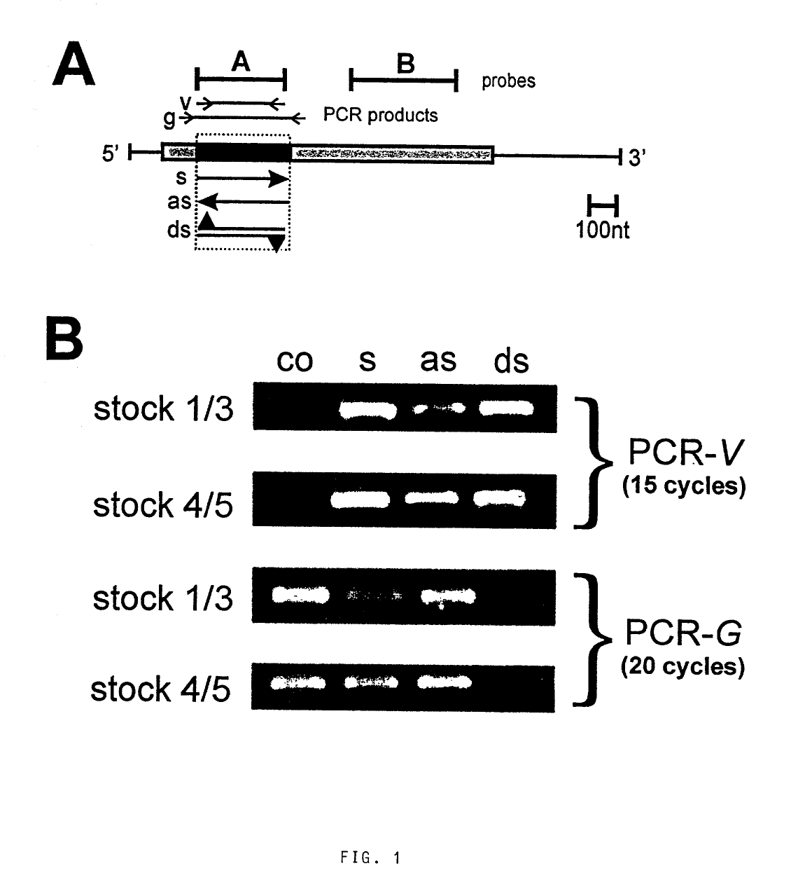

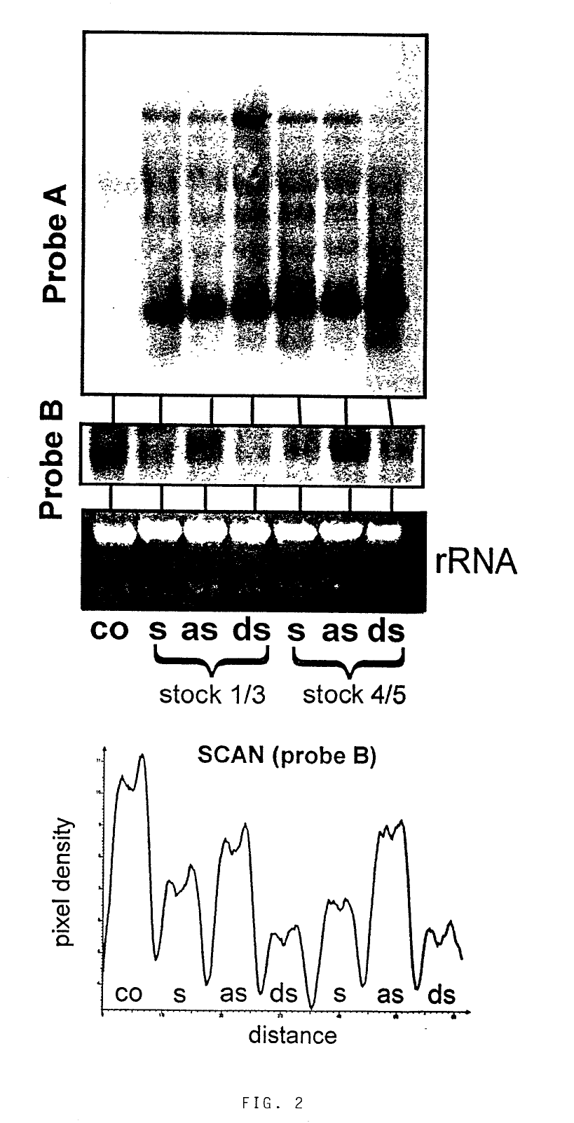

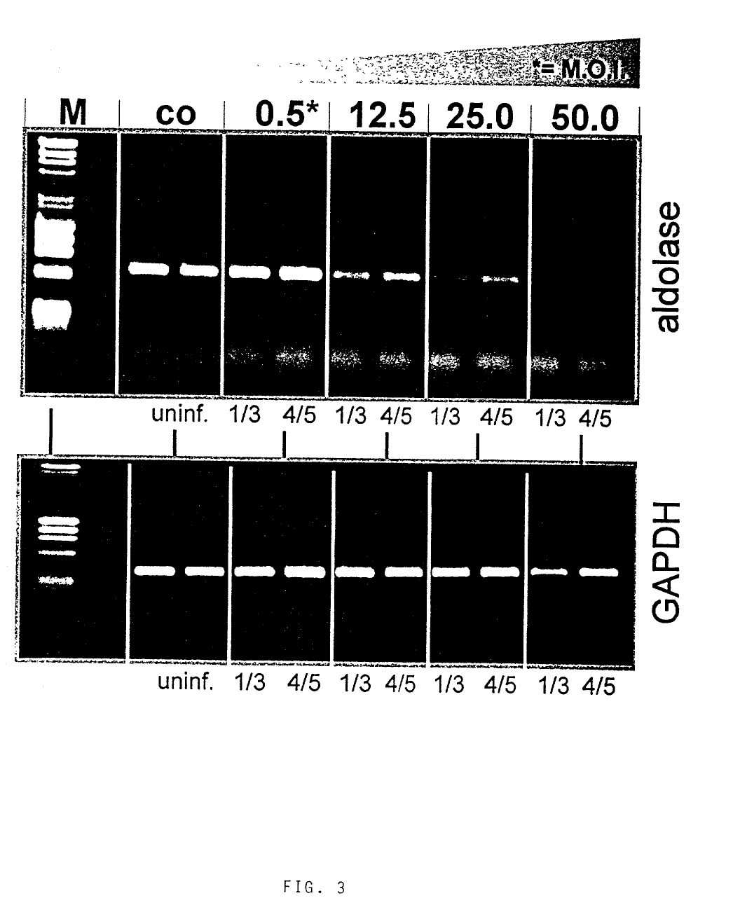

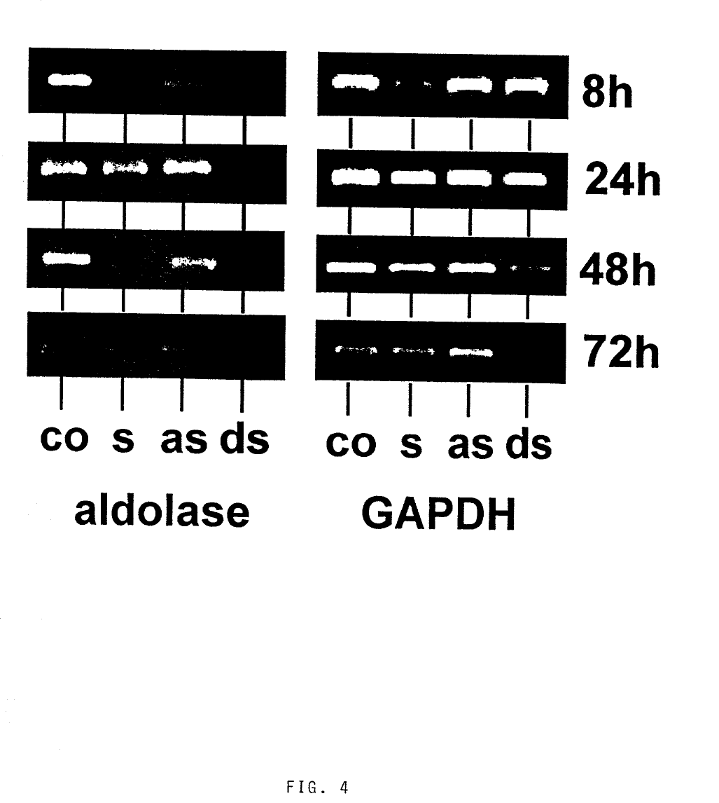

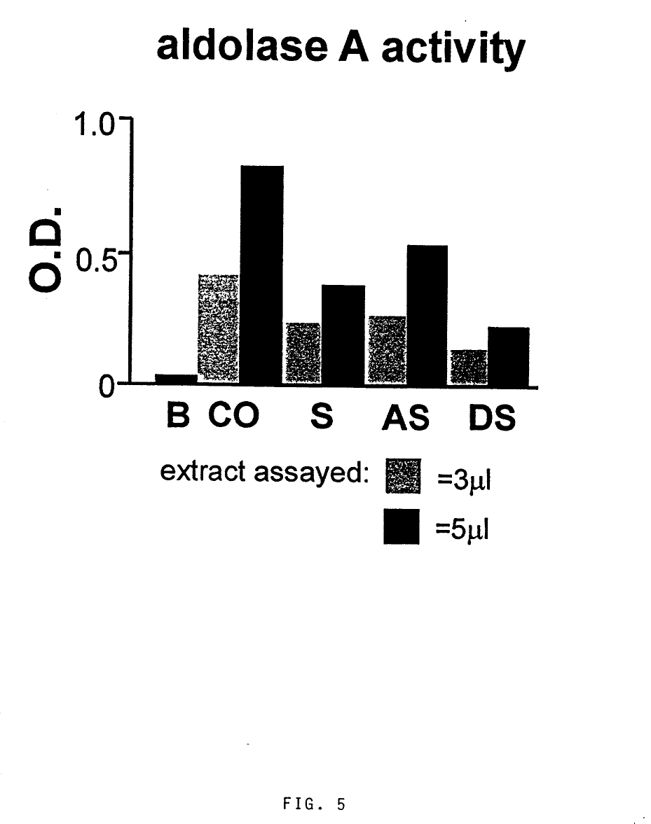

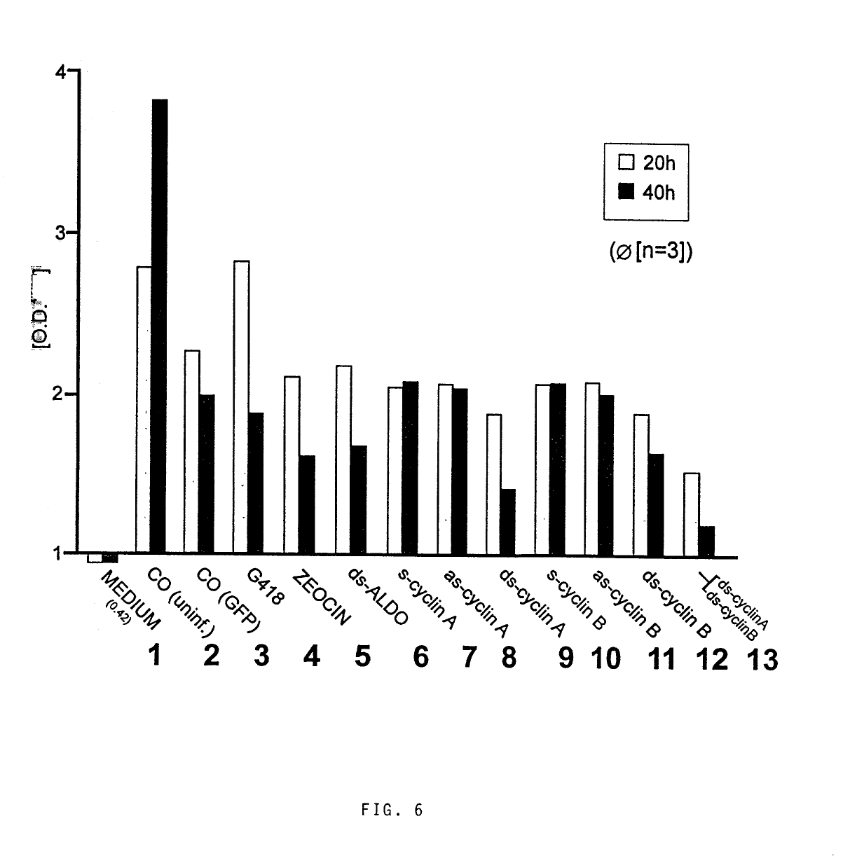

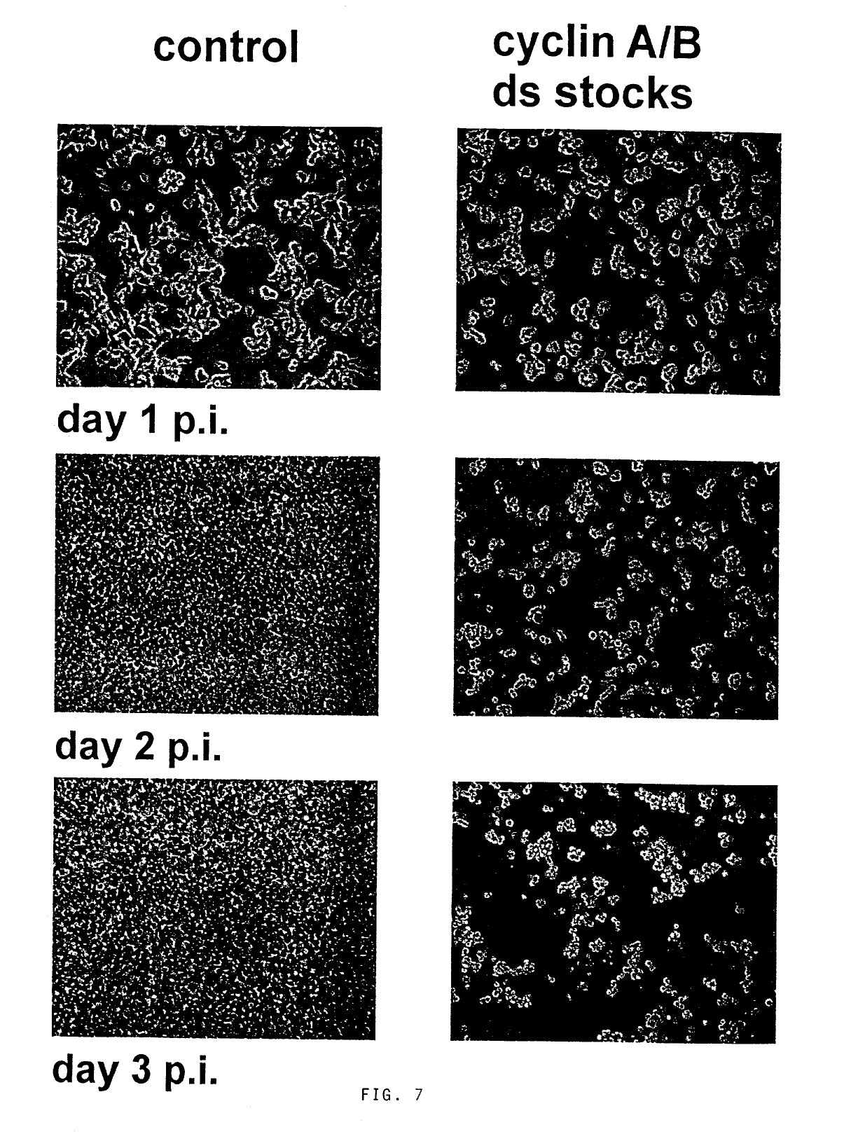

FIG. 2: Analysis of aldolase RNA by Northern blots. Top panel: Total RNA of infected cells (see FIG. 1) was separated by gel analysis transferred to a membrane and probed either with labeled A which covers the virally expressed region or with B that is specific for chromosomal copies of aldolase A RNA. Probe A required 1.5 h of exposure and B overnight exposure to x-ray film. At the bottom is shown a scan of the autoradiograph of the probe B blot.

FIG. 3: Correlation between inhibition of gene transcription and virus titration.

Cells were infected with a M.O.I. (multiplicity of infection) of 0.5 to 50 with s/as virus block stocks and incubated for 24 hours. Total RNA was isolated and converted into cDNA. PCR was carried out for 25 cycles either with aldolase or GAPDH (control) specific primers. The amplicons were visualized by conventional agarose electrophoresis. The results with two independent virus block stocks are shown (⅓ and ⅘).

FIG. 4: Kinetics of inhibition by as/s virus stocks. HEK (Human embryonic kidney) cells were infected with ⅓ block stocks or the individual s and as stocks. The cells were incubated for the times indicated followed by PCR analysis of the transcript levels.

FIG. 5: Measurement of aldolase A enzyme activity.

Co indicates the enzyme level in unifected cells and B is a buffer, negative control, as, s and block stocks (ds) were used to infect the cells at MOI of 25 for 24 hours. Cells were harvested, lysed and the enzyme activity was measured using a commercial assay and either 3 or 5 μl lysate.

FIG. 6: Cell cycle arrest by cyclin down-regulation.

From left to right: 1. medium control; 2. uninfected cells (maximal proliferation); 3. cells infected with a virus expressing green fluorescent protein (GFP, infection control); 4. and 5. assay control with antibiotics G418 and zeocin; 6. human aldolase A dsRNA (inhibition control); 7. cyclin A sense SFV; ; 8. cyclin A antisense SFV;; 9. cyclin A sense and antisense SFV (ds); 10. cyclin B sense SFV; ; 11. cyclin B antisense SFV; 12. cyclin B sense and antisense SFV (ds); 13. cyclin A and cyclin B sense and antisense SFV (ds).

FIG. 7: Microscopic image of culture of cells infected with virus expressing GFP and culture of cyclin A and cyclin B sense and antisense SFV.

### DETAILED DESCRIPTION OF THE INVENTION

The expression “ds RNA” as used herein means double stranded RNA.

The expression “ss RNA” as used herein means single stranded RNA.

The term “sense” as used herein means a RNA sequence complementary to a strand of the mRNA of the target gene.

The term “anti-sense” as used herein means a RNA sequence complementary to the sense strand.

The term “sequence specific for” as used herein means that the sequence of the sense strand and of the anti-sense RNA strand has at least 90%, preferably 95%, more preferably 99% and most preferably 100%, bases identical to the target gene.

The process of the present invention for inhibiting the expression of a target gene in cells or tissue comprises infection of said cells or tissue with (a) viral particles consisting essentially of single stranded ribonucleic acid (ss RNA) which expresses sense RNA strand; and (b) viral particles consisting essentially of single stranded ribonucleic acid (ss RNA) which expresses anti-sense RNA strand; wherein the sense and anti-sense RNA strands comprise homologous nucleotide sequences to a portion of the target gene.

The present invention is useful for selective inhibition of specific gene functions by biologic generation of ds RNAs in the cells. The present invention may be useful for the treatment or the prevention of specific diseases or pathologies by inhibition of specific over-expressed genes, which are required for the initiation or the maintenance of the diseases or pathologies. Treatment would include amelioration of any symptoms associated with the disease or clinical indications associated with the pathology.

For example, the present invention may be useful for treatment or prevention of suffering from tumors by inhibition of specific gene function in patients. Tumors include ovary, prostate, breast, colon, liver, stomach, brain, head-and-neck and lung cancers.

Another use of the present invention could be a method to identify gene function in an organism by specific inhibition of expression.

Furthermore, the present invention may be useful for analysis or prevention of the mechanism for growth, development, metabolism, disease resistance or other biological processes.

The present invention provides for the biological generation of ds RNAs into cells or tissue with ease. It also provides highly efficient amplification of the introduced ss RNAs, stability of ds RNAs in cells and tissue, efficient inhibition of gene expression and biological safety.

The term “alphavirus” has its conventional meaning in the art, and includes the various species of alphaviruses such as Eastern Equine Encephalitis virus (EEE), Venezuelan Equine Encephalitis virus (VEE), Everglades virus, Mucambo virus, Pixuna virus, Western Equine Encephalitis virus (WEE), Sindbis virus, South African Arbovirus No. 86, Semliki Forest virus, Middelburg virus, Chikungunya virus, O'nyong-nyong virus, Ross River virus, Barmah Forest virus, Getah virus, Sagiyama virus, Bebaru virus, Mayaro virus, Una virus, Aura virus, Whataroa virus, Babanki virus, Kyzylagach virus, Highlands J virus, Fort Morgan virus, Ndumu virus, and Buggy Creek virus. The term “alphavirus” also includes vectors derived thereof. The preferred alphavirus include Semliki Forest Virus (SFV) (Liljeström and Garoff, 1991). A new generation of animal cell expression vectors based on the Semliki Forest virus replicon, Bio/Technology 9, 1356-1361), Sindbis Virus (SIN)(Xiong et al., 1989 Sindbis virus: an efficient broad host range vector for gene expression in animal cells, Science 243, 1188-1191) and Venezuelan Equine Encephalitis Virus (VEE) (Davis et al., 1989 In vitro synthesis of infectious Venezuelan equine encephalitis virus RNA from a cDNA clone: analysis of a viable deletion mutant, Virology 171, 189-204), for example. The alphavirus and vectors derived thereof are well-known in the art and commercially available.

As is known, alphaviruses are RNA viruses that can mediate efficient cytoplasmic gene expression in vertebrate cells. Due to engineering of alphavirus replication machinery, the alphavirus can facilitate high-level expression of heterologous RNAs and proteins.

In the process of the present invention, cells or tissue are infected with an amount of viral particles comprising ss RNA, which allows delivery of at least one copy per cell. As disclosed herein, the infection is made with a number superior or equal to 10 viral particles per cell.

The infection procedure is well known in the art. The in vitro infection in cell lines and primary cell cultures, like fibroblasts, hepatocytes, neurons, for example, is carried out by addition of SFV viral particles directly to the cell cultures. The viral particles will recognize receptors on the cell surface, penetrate the cell membrane either by fusion or endocytosis (depending on cell type), where after the RNA molecules will be liberated into the cytoplasm (“The Alphaviruses: Gene Expression, Replication, and Evolution, Strauss”, J. H and Strauss, E. G., 1994, Microbiological Reviews 58, 491-562).

The in vivo infection requires injection of the SFV viral particles to the target tissue. Injection of SFV viral particles (“Efficient in vivo expression of a reporter gene in rat brain after injection of recombinant replication-deficient Semliki Forest virus”, Lundstrom, K., Grayson, J. R., Pink J. R. and Jenck, F., 1999, Gene Therapy & Molecular Biology 3, 15-23) will result in a similar infection procedure as described for the in vitro situation above.

Cells or tissue in the present process are infected with separate viral particles expressing complementary strands, i.e., one set of viral particles expresses sense RNA strands and the other set of viral particle expresses anti-sense RNA strands.

As disclosed herein, cells or tissue in the present process may be co-infected with equal amounts of viral particles consisting essentially of ss RNA which expresses sense RNA strand and of viral particles consisting essentially of ss RNA which expresses anti-sense RNA strand, respectively, to allow the formation of ds RNAs capable of interfering with gene expression. Higher doses of ds RNA may yield more effective inhibition.

In the present invention a viral particle encompasses a ss RNA strand comprising a homologous nucleotide sequence to a portion of the target gene, and the vector of the alphavirus into which the ss RNA is cloned. The ss RNA strand may be cloned either in sense or anti-sense orientation into the said vector. The other genes present in the vector are the nonstructural alphavirus genes especially the nsP-1-4 genes (The Alphaviruses: Gene Expression, Replication, and Evolution, Strauss, J. H and Strauss, E. G., 1994, Microbiological Reviews 58, 491-562), responsible for RNA replication in host cells. Expression of nsP1-4 results in the formation of the replicase complex, that will initiate extensive RNA replication, i.e. generation of large numbers of sense and anti-sense RNA, capable of efficient ds RNA formation.

The term “viral particles consisting essentially of ss RNA which expresses a sense RNA strand” means that the viral particles comprise essentially ss RNA which expresses a sense RNA strand, rather than ss RNA which expresses anti-sense RNA. The term “viral particles consisting essentially of ss RNA which expresses an anti-sense strand” means that the viral particles comprise essentially ss RNA which expresses anti-sense RNA, rather than ss RNA which expresses sense RNA.

The process described herein allows in general inhibition of many different types of target genes in eukaryotic cells or tissue. The target gene may be a eukaryotic gene, a viral gene, a gene of a pathogen or a synthetic gene. Clearly, the target gene may be a gene derived from the cell, i.e. a cellular gene, a transgene, i.e. a gene construct inserted at an ectopic site in the genome of the cell or a gene from a pathogen, capable of infecting an organism from which the cell is derived.

The target genes may be any gene of interest, there already having been a large number of proteins of interest identified and isolated. The target gene may be a developmental gene, like cyclin kinase inhibitors, growth/differentiation factors and their receptors, telomerase reverse transcriptase (TERT), an oncogene, a tumor suppressor gene or an enzyme, for example. A gene derived from any pathogen may be the target of inhibition.

Since inhibition in the present invention is sequence specific, sense and anti-sense RNA strands introduced into the cells or tissue comprise a complementary nucleotide sequence of a portion of the target gene.

A complete homology between the RNA and the target gene is not required to practice the present invention. As disclosed herein, sequence variations due to genetic mutations, polymorphisms, or evolutionary divergences, for example, are tolerated. RNA strands with insertions, deletions, and single point mutations to the target gene have been found to be effective for inhibitions.

The length of the said homologous nucleotide sequence should be at least 50 bases, preferably 75, 100 or 125 bases.

In the process of the present invention, the inhibition of the target gene expression demonstrates a loss of phenotype. Depending on the target gene and the intracellular dose of ds RNA, the process of the present invention may result in partial or complete loss of function of the target gene in the cells or tissue of the organism.

Inhibition of gene expression refers to the absence or the decrease in the level of protein and/or mRNA from a target gene. The consequences of inhibition may be assayed for properties of the cell or organism by molecular biology methods such as RNA solution hybridization, Northern hybridization (Sambrook et al., Molecular Cloning, vol. 1, 7.37 & 7.39) and biochemical assays like enzyme linked immunoabsorbent assay (ELISA), Western blotting (Towbin et al. 1979; Bunette 1981 or Sambrook et al., Molecular Cloning, vol. 3, 18.60) or radioimmunoassay (RIA) (Sambrook et al., Molecular Cloning, vol. 3, 18.19-18.20), for example.

The degree of inhibition may be estimated by comparing the values from untreated cells to those obtained from cells treated according to the method of the present invention.

The present invention concerns also any cell containing two complementary RNA strands, a sense and an anti-sense RNA strand, which form a double stranded RNA inside the said cell, and because of nucleotide sequence homology to a portion of a specific target gene are capable of interfering with the expression of the said target gene.

As disclosed herein, the eukaryotic cells or tissue with the target gene may be any cell or tissue type, which can be infected by an alphavirus. They may be from the vascular or extravascular circulation, from the blood or lymph system, from muscles, liver, brain, or from the cerebrospinal fluid, for example.

The eukaryotic cells or tissue may be contained in any organism including fish, amphibians, reptilians, insects or mammal like cattle, pig, hamster, mouse, rat, primate and human, for example.

Furthermore, the present invention provides a kit comprising reagents to inhibit expression of a target gene, wherein the kit comprises at least a sufficient amount of single stranded RNA viral particles which expresses sense RNA strand and of ss RNA viral particles which expresses anti-sense RNA strand, wherein the sense and anti-sense strands are complementary to each other and form a ds RNA comprising a homologous nucleotide sequence to a portion of said target gene and are capable of interfering with the expression of the said target.

Such a kit may include reagents necessary to carry out the in vivo or in vitro delivery of RNA to test samples or subjects.

Such a kit may also include instructions to allow a user of the kit to practice the invention.

To treat a disease or pathologic condition, a target gene may be selected which is expressed during the development of the disease or which is the cause of the pathologic condition.

To prevent a disease or a pathologic condition, a target gene may be selected which is required for initiation and/or maintenance of the disease or the pathologic condition.

The present invention maybe used for treatment or prevention of cancer including solid tumors or leukemias, by co-infection of tumors with vectors which are viral particles consisting essentially of ss RNA which expresses a sense RNA strand and viral particles consisting essentially of ss RNA which expresses an anti-sense strand, with the aim of generating ds RNA for the inhibition of mRNA translation of a gene required for the maintenance of the carcinogenic/tumorigenic phenotype.

The present invention may be used for treatment or prevention of infectious diseases due to a pathogen, for example. Cells or tissue infected or which may be infected by human immunodeficiency virus (HIV) may be targeted according to the present invention in order to inhibit the expression of a specific gene responsible or required for initiation and/or maintenance of said infection.

The present invention concerns also the use of (a) viral particles consisting essentially of single stranded ribonudeic acid (ss RNA) which expresses sense RNA strand, and (b) viral particles consisting essentially of single stranded ribonucleic acid (ss RNA) which expresses anti-sense RNA strand, wherein the sense and anti-sense RNA strands comprise homologous nucleotide sequences of a portion of a target gene, for the preparation of a medicament for treating diseases.

Moreover, the present invention concerns viral particles consisting essentially of single stranded ribonucleic acid (ss RNA) which expresses sense RNA strand, and viral particles consisting essentially of single stranded ribonucleic acid (ss RNA) which expresses anti-sense RNA strand, wherein the sense and anti-sense RNA strands comprise homologous nucleotide sequences of a portion of a target gene, for use as a therapeutic active substance for the treatment or prevention of disease, in particular as anti-cancer substance.

The present invention also concerns a composition comprising (a) viral particles consisting essentially of single stranded ribonucleic acid (ss RNA) which expresses sense RNA strand, and (b) viral particles consisting essentially of single stranded ribonucleic acid (ss RNA) which expresses anti-sense RNA strand, wherein the sense and anti-sense RNA strands have homologous nucleotide sequences to a portion of a target gene, for the inhibition of the expression of the said target gene in cells or tissue. A pharmaceutical composition optionally includes pharmaceutically acceptable excipients.

The following examples are provided by way of illustration and not by way of limitation.

In the examples below the methods and techniques required are known from the literature and are described, for example, in Sambrook et al., 1989.

In the examples below, the SFV vector used is a noncytopathogenic version with two point mutations in the SFV nonstructural gene nsP2 (Ser259Pro and Arg650Asp) described by Lundstrom, K., Schweitzer, C., Richards, J. G., Ehrengruber, M. U., Jenck, F. and Muelhardt, C., Gene Therapy and Molecular Biology, 4, 23-31, 1999), Semliki Forest virus vectors for in vitro and in vivo applications. This modified SFV vector does not inhibit the endogenous gene expression in the infected host cells, which allows targeted and specific gene inhibition by the dsRNA technology.

### EXAMPLES

### Example 1

Inhibition of Aldolase A expression in BHK (Baby hamster kidney) cells (ATCC registered number: CCL-10) (FIG. 1.).

Based on the human aldolase A gene (M. Sakakibara, T. Mukai & K. Hori, Nucleotide sequence of a cDNA clone for human aldolase: a messenger RNA in the liver, Biochem. Biophys. Res. Commun. 30, 413-420, 1985 which is incorporated herein by reference) 3 pairs of oligonucleotide primers were selected to amplify the required gene regions. VA (nt 210-240 as described by M. Sakakibara, T. Mukai & K. Hori, Nucleotide sequence of a cDNA clone for human aldolase: a messenger RNA in the liver, Biochem. Biophys. Res. Commun. 30, 413-420, 1985 which is incorporated herein by reference) and VB (nt 740-710 as described by M. Sakakibara, T. Mukai & K. Hori, Nucleotide sequence of a cDNA clone for human aldolase: a messenger RNA in the liver, Biochem. Biophys. Res. Commun. 30, 413-420, 1985 which is incorporated herein by reference) amplify a region of about 600 nucleotides used for construction of the sense and antisense virus stocks. GA (nt 170-200 as described by M. Sakakibara, T. Mukai & K. Hori, Nucleotide sequence of a cDNA clone for human aldolase: a messenger RNA in the liver, Biochem. Biophys. Res. Commun. 30, 413-420, 1985 which is incorporated herein by reference) and GB (nt 780-750 as described by M. Sakakibara, T. Mukai & K. Hori, Nucleotide sequence of a cDNA clone for human aldolase: a messenger RNA in the liver, Biochem. Biophys. Res. Commun. 30, 413-420, 1985 which is incorporated herein by reference) amplify a chromosomal region of the aldolase gene. The articles incorporated herein by reference are incorporated to the extent of the sequences here mentioned. Northern Probe A is generated using primers VA and VB and probe B was amplified with a primer pair of the upstream region (nt 951-980 and nt 1330-1301 as described by M. Sakakibara, T. Mukai & K. Hori, Nucleotide sequence of a cDNA clone for human aldolase: a messenger RNA in the liver, Biochem. Biophys. Res. Commun. 30, 413-420, 1985). Cells were infected and grown for 24 hours. RNA was isolated and converted into cDNA according to standard procedures. All PCR products were subcloned into common cloning vectors for sequencing. The VA/VB was further cloned into the SFV vector to generate infectious SFV particles. The virally encoded aldolase mRNA is abundant and detected after 15 cycles of PCR in virus infected cells. No signal is obtained in cells without virus. Using the genomic primers for aldolase mRNA a band of the expected size is amplified in the uninfected cells and cells infected with sense or antisense producing viruses. The mixture of both the sense and antisense viruses is a potent inhibitor of expression of the chromosomal aldolase gene whilst the viral gene expression remains unaffected.

### Example 2

Analysis of aldolase RNA by Northern blots (FIG. 2).

Total RNA from either uninfected cells or cells infected with the virus stocks indicated was separated on a standard formamide gel, transferred after electrophoresis to a nitrocellulose membrane and then probed either with radiolabeled fragment A or B (see example 1). Probe A detects almost exclusively the virus-derived aldolase RNA due to the short exposure time of 45 minutes. Probe B detects only chromosomally encoded aldolase mRNA after 16 hours exposure of the hybrized blot to film. A stained gel with ribosomal RNA was used to control loading (below probe B). Especially in the gel scan, it is evident that the aldolase mRNA levels are lowest in the cells infected with both viruses.

### Example 3

The inhibition of gene transcription is dependent on virus titers.

As it can be seen in FIG. 3, relative to the control, the levels of aldolase mRNA start to decrease at a M.O.I. of 12.5 and at 50 essentially no mRNA can be detected using this sensitive assay. The levels of another chromosomal control gene (GAPDH) are not altered with increasing M.O.I.

### Example 4

Kinetics of inhibition by as/s virus stocks.

BHK cells (ATCC registered number: CCL-10) were infected with as or s or an as/s mix of aldolase RNA virus stocks. At the time points indicated in the figure, RNA was isolated and converted into cDNA. After PCR the products were analyzed by agarose gel electrophoresis. At 8 hours marginal destruction of genomic aldolase RNA is evident and the highest activity is detectable at 48 hours. In this particular experiment also the sense expressing virus influenced RNA stability. The GAPDH RNA remains unaltered except in the 48 and 72 h samples, in which a reduction of the RNA levels is evident in cells infected with the s/as virus mix. This is probably related to cell death because aldolase is an essential enzyme.

### Example 5

Reduction of aldolase enzyme activity by s/as aldolase virus stocks

BHK cells (ATCC registered number: CCL-10) were infected with the stocks indicated and grown for 24 hours under standard cell culture conditions. The cells were harvested and lysed in 1×PBS containing 0.2% Triton X-100. After centrifugation for 10 min at 16'000 g and 4° C. the supernatant was recovered and either 3 μl (grey bars) or 5 μl (black bars) were assayed using a commercial kit (SIGMA, catalogue #: 752-A) and the protocol supplied. The most significant reduction of enzyme activity is as expected in the sample infected with both the s and as virus stocks.

### Example 6

Cyclin “knock down” results in cell cycle arrest

Cell cycle arrest by cyclin down-regulation. Human embryonic kidney (HEK293) cells (ATCC registered number: CRL-1573) were infected with the SFV virus particles indicated at time point zero and proliferation was assayed after 20 (light grey bars) and 40 hours (dark grey bars) in culture using a commercial color assay (Promega G5421 according to technical bulletin TB245). The mixture of the cyclin A and B blocking virus stocks was most efficient and even more potent than inhibition of cell growth by antibiotics (neomycin and zeocin).

The sequence of the cyclin A is those described in (“Hepatitis B virus integration in a cyclin A gene in a hepatocellular carcinoma”, Wang, Chevenisse X., Henglein B., Brechot C., Nature 343:555-557(1990)) and the sequence of the cyclin B is that described by (Kim D. G., Choi S. S., Kang Y. S., Lee K. H., Kim U. -J., Shin H. -S., Submitted (MAY 6, 1997) to the EMBL ACC-NO AF002822/GenBank/DDBJ databases, Life Science, Pohang University of Science and Technology, San 31, Pohang, Kyungbuk 790-784, Korea).

### Example 7

Culturing of cells infected with sense and anti-sense in one vector

Sense and anti-sense fragments of the cyclin A and B genes were cloned into a single SFV vectors by the introduction of a second subgenomic 26S promoter. The constructs were the following:

SFV 26S-sense cyclin A-SFV 26S-anti-sense cyclin A and

SFV 26S-sense cyclin B-SFV 26S-anti-sense cyclin B

Infections of HEK293 cells with SFV-cyclin A or SFV-cyclin B alone, or together, did not result in any arrest of cell proliferation.

This indicated that constructs with both sense and anti-sense fragments in the same vector are not able to inhibit expression of chromosomal cyclin genes.

What is claimed is:

1. A process for inhibiting expression of a target gene in cells or tissue, comprising infecting said cells or tissue with viral particles consisting essentially of single stranded ribonucleic acid (ss RNA) which expresses a sense RNA strand, and viral particles consisting essentially of ss RNA which expresses an anti-sense RNA strand, wherein the sense and anti-sense RNA strands comprise homologous nucleotide sequences to a portion of said target gene.

2. The process of claim 1 wherein said cells or tissue are/is infected with equal amounts of viral particles consisting essentially of ss RNA expressing sense RNA strand and of viral particles consisting essentially of ss RNA expressing anti-sense RNA strand.

3. The process of claim 1 wherein ss RNA is cloned into the vector of the alphavirus in sense orientation to provide viral particles consisting essentially of ss RNA which expresses a sense RNA strand, and ss RNA is cloned into the vector of the alphavirus in anti-sense orientation to provide viral particles consisting essentially of ss RNA which expresses an anti-sense RNA strand.

4. The process of claim 1 in which said target gene is an eukaryotic gene, a viral gene or a synthetic gene.

5. The process of claim 1 in which said target gene is a developmental gene, an oncogene, a tumor suppressor gene or an enzyme.

6. The process of claim 1 in which said homologous nucleotide sequence is specific for said target gene and is at least 50 bases in length.

7. A kit comprising reagents to inhibit the expression of a target gene in cells or tissue, wherein said reagents comprise viral particles consisting essentially of single stranded RNA (ss RNA) which expresses a sense RNA strand and viral particles consisting essentially of ss RNA which expresses an anti-sense RNA strand, wherein said sense RNA strand and said anti-sense RNA strand are complementary and form inside said cells or tissue a ds RNA comprising a homologous nucleotide sequence to a portion of said target gene such that said reagents are capable of interfering with expression of said target gene.

8. A composition comprising viral particles consisting essentially of single stranded ribonucleic acid (ss RNA) which expresses sense RNA strand, and viral particles consisting essentially of single stranded ribonucleic acid (ss RNA) which expresses anti-sense RNA strand, wherein the sense and anti-sense RNA strands comprise homologous nucleotide sequences to a portion of a target gene.


SEQUENCE LISTING (3 sequences)

SEQ ID NO: 1 - RNA, 1299 nt, Homo sapiens
auguugggcaacucugcgccggggccugcgacccgcgaggcgggcucggcgcugcuagcauugcagcagacggcgcuccaagaggaccaggagaauaucaacccggaaaaggcagcgcccguccaacaaccgcggacccgggccgcgcuggcgguacugaaguccgggaacccgcggggucuagcgcagcagcagaggccgaagacgagacggguugcaccccuuaaggaucuuccuguaaaugaugagcaugucaccguuccuccuuggaaagcaaacaguaaacagccugcguucaccauucauguggauccagcagaaaaagaagcucagaagaagccagcugaaucucaaaaaauagagcgugaagaugcccuggcuuuuaauucagccauuaguuuaccuggacccagaaaaccauuggucccucuugauuauccaauggaugguaguuuugagucaccacauacuauggacaugucaauuguauuagaagaugaaaagccagugaguguuaaugaaguaccagacuaccaugaggauauucacacauaccuuagggaaauggagguuaaauguaaaccuaaaguggguuacaugaagaaacagccagacaucacuaacaguaugagagcuauccucguggacugguuaguugaaguaggagaagaauauaaacuacagaaugagacccugcauuuggcugugaacuacauugauagguuccugucuuccaugucagugcugagaggaaaacuucagcuugugggcacugcugcuaugcuguuagccucaaaguuugaagaaauauaccccccagaaguagcagaguuuguguacauuacagaugauaccuacaccaagaaacaaguucugagaauggagcaucuaguuuugaaaguccuuacuuuugacuuagcugcuccaacaguaaaucaguuucuuacccaauacuuucugcaucagcagccugcaaacugcaaaguugaaaguuuagcaauguuuuugggagaauuaaguuugauagaugcugacccauaccucaaguauuugccaucaguuauugcuggauccgccuuucauuuagcacucuacacagucacgggacaaagcuggccugaaucauuaauacgaaagacuggauauacccuggaaagucuuaagccuugucucauggaccuucaccagaccuaccucaaagcaccacagcaugcacaacagucaauaagagaaaaguacaaaaauucaaaguaucaugguguuucucuccucaacccaccagagacacuaaaucuguaa

SEQ ID NO: 2 - RNA, 1197 nt, homo sapiens
auggcgcugcuccgacgcccgacgguguccagugauuuggagaauauugacacaggaguuaauucuaaaguuaagagucaugugacuauuaggcgaacuguuuuagaagaaauuggaaauagaguuacaaccagagcagcacaaguagcuaagaaagcucagaacaccaaaguuccaguucaacccaccaaaacaacaaaugucaacaaacaacugaaaccuacugcuucugucaaaccaguacagauggaaaaguuggcuccaaaggguccuucucccacaccugaggaugucuccaugaaggaagagaaucucugccaagcuuuuucugaugccuugcucugcaaaaucgaggacauugauaacgaagauugggagaacccucagcucugcagugacuacguuaaggauaucuaucaguaucucaggcagcuggagguuuugcaguccauaaacccacauuucuuagauggaagagauauaaauggacgcaugcgugccauccuaguggauccgcugguacaaguccacuccaaguuuaggcuucugcaggagacucuguacaugugcguuggcauuauggaucgauuuuuacagguucagccaguuucccggaagaagcuucaauuaguugggauuacugcucugcucuuggcuuccaaguaugaggagauguuuucuccaaauauugaagacuuuguuuacaucacagacaaugcuuauaccaguucccaaauccgagaaauggaaacucuaauuuugaaagaauugaaauuugaguugggucgacccuugccacuacacuucuuaaggcgagcaucaaaagccggggagguugauguugaacagcacacuuuagccaaguauuugauggagcugacucucaucgacuaugauauggugcauuaucauccuucuaagguagcagcagcugcuuccugcuugucucagaaggauccaggacaaggaaaauggaacuuaaagcagcaguauuacacaggauacacagagaaugaaguauuggaagucaugcagcacauggccaagaauguggugaaaguaaaugaaaacuuaacuaaauucaucgccaucaagaauaaguaugcaagcagcaaacuccugaagaucagcaugaucccucagcugaacucaaaagccgucaaagaccuugccuccccacugauaggaagguccuag

SEQ ID NO: 3 - DNA, 10610 bp, Artificial Sequence
  pSFV2gen(PD) vector
gatggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaggctttgagcgagagcgccactatggtgtacaacgaaagggagttcgtcaacaggaaactataccatattgccgttcacggaccctcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgcttgccatcccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcgacgtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctggagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggctgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattggatcccggtccgaagcgcgctttcccgggaactcgagttcactagtcgatcccgcggccgctttcgaacctaggcaagcatgcgggcccagtgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaaactagaaatcgcgatttctagtctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcgcgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttctgtctaagcggatgccgggagcagacaagcccgtcagggcgcgtcagcgggtgttggcgggtgtcggggctggcttaactatgcggcatcagagcagattgtactgagagtgcaccatatcgacgctctcccttatgcgactcctgcattaggaagcagcccagtactaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccggggtgcggaacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttacctcacactata